(19) United States
(12) Patent Application Publication
(10) Pub. No.: US 2023/0233767 A1
(43) Pub. Date: Jul. 27, 2023

(54) A SEALABLE JOINT

(71) Applicant: CONSORT MEDICAL LIMITED, King's Lynn Norfolk (GB)

(72) Inventors: George SAVELL, King's Lynn Norfolk (GB); Hugh PERKINS, King's Lynn Norfolk (GB); Deborah NORRIS, King's Lynn Norfolk (GB)

(21) Appl. No.: 18/013,719

(22) PCT Filed: Jul. 2, 2021
(86) PCT No.: PCT/GB2021/051681

§ 371 Date: Dec. 29, 2022

(30) Foreign Application Priority Data
Jul. 3, 2020 (GB) 2010250.5

(51) Int. Cl. A61M 5/20; F16J 15/3284

(52) U.S. Cl. CPC A61M 5/2046; F16J 15/3284

(57) ABSTRACT

A sealable joint including a first component, a second component, and a third component. The first component includes a stem having a bore therethrough for the passage of a fluid. The second component is disposed on the first component, and the third component includes a sealing surface and one or more radially inwardly projecting members. One of the second component and the first component, or a combination of the second component and the first component includes a first flange and a second flange that defines an annular recess therebetween. In a sealing configuration, the second component is received by the third component to form a sealed joint in which at least part of the first flange deforms against the sealing surface, the one or more radially inwardly projecting members are disposed in the annular recess between the first flange and the second flange.

This invention relates to a sealable joint, and particularly but not exclusively to a sealable joint that may be used in a medical device such as a medicament delivery device.

### BACKGROUND

Certain sealable joints are required to provide a fluidic seal whilst mechanically linking two components together. A known example of such a sealable joint includes a compression fitting that that incorporates an outer compression nut and an inner compression ring or ferrule. The inner compression ring may be compressed within the fitting in order to provide fluidic sealing.

Known sealable joints suffer from certain drawbacks, however. Certain sealable joints may be prone to failure of either or both of the fluidic seal or mechanical linkage of the two (or more) components under certain conditions.

It is an object of certain embodiments of the present invention to provide an improved sealable joint. The sealable joint may offer improved sealing and/or mechanical retention under conditions such as creep or the impact of aging, vibration (sinusoidal and/or random), pressure loading, axial loading, transverse loading, and/or shock loading.

### BRIEF SUMMARY OF THE DISCLOSURE

In accordance with an aspect of the present invention there is provided a sealable joint comprising a first component, a second component, and a third component;

the first component comprising a stem having a bore therethrough for the passage of a fluid;

the second component being disposed on the first component; and

the third component comprising a sealing surface and one or more radially inwardly projecting members; and

wherein one of the second component and the first component, or a combination of the second component and the first component comprise a first flange and a second flange that define an annular recess therebetween;

wherein in a sealing configuration, the second component is received by (e.g. in) the third component to form a sealed joint in which at least part of the first flange deforms against the sealing surface, the one or more radially inwardly projecting members are disposed in the annular recess between the first flange and the second flange, and an outer surface of the second flange is radially outward of at least part of the one or more radially inwardly projecting members; and

wherein in the sealing configuration fluid may flow through the bore from a first side of the sealed joint to a second side of the sealed joint.

In certain embodiments, the second component may be affixed on an external surface of the first component. The second component may comprise a more resilient material relative to the first component.

In the sealing configuration, the at least part of the first flange may form a sealed interference fit with the sealing surface.

In certain embodiments, the one or more radially inwardly projecting members comprise a plurality of radially flexible fingers.

In certain embodiments, the outer surface of the second flange may narrow relative to a longitudinal axis of the sealable joint.

In certain embodiments, the second flange may comprise an abutment surface that faces the one or more radially inwardly projecting members in the sealing configuration, wherein abutment between the abutment surface and the one or more radially inwardly projecting members may limit axial movement of the second flange relative to the third component.

In certain embodiments, the first component may comprise a third flange, wherein the third flange is on a side of the first flange that is opposite to the second flange such that the first flange is between the second flange and the third flange, and wherein abutment between the third flange and the third component may limit axial movement of the first component relative to the third component.

In certain embodiments, the first component and the second component form a single unitary part.

In accordance with another aspect of the present invention, there is provided an autoinjector sub-assembly comprising a sealable joint as described above.

The autoinjector sub-assembly may comprise a propellant source, wherein the sealable joint may seal the propellant source to another component of the auto-injector sub-assembly. The propellant source may comprise a propellant housing defining a reservoir for containing a propellant, and the first component may be moveable relative to the propellant housing so as to selectively put the bore in fluid communication with the reservoir.

The third component may form at least part of the other component of the auto-injector sub-assembly.

In accordance with another aspect of the present invention, there is provided a propellant source comprising a propellant housing defining a reservoir for containing a propellant, a first component comprising a stem having a bore therethrough, and a second component being disposed on the first component, wherein one of the second component and the first component, or a combination of the second component and the first component comprise a first flange and a second flange that define an annular recess therebetween, and wherein the first component is moveable relative to the propellant housing so as to selectively put the bore in fluid communication with the reservoir.

In certain embodiments, the second component may be affixed on an external surface of the first component. The second component may comprise a more resilient material relative to the first component.

In certain embodiments, the outer surface of the second flange may narrow relative to a longitudinal axis of the propellant source.

In certain embodiments, the first component may comprise a third flange, wherein the third flange is on a side of the first flange that is opposite the second flange.

In certain embodiments, the first component and the second component form a single unitary part.

In accordance with an embodiment of the present invention, there is provided a sealable joint comprising a male connector part, and a female connector part;

the male connector part comprising a stem having a bore therethrough for the passage of a fluid;

the female connector part comprising a sealing surface and one or more radially inwardly projecting members; and

wherein male connector part comprises a first flange and a second flange that define an annular recess therebetween;

wherein in a sealing configuration, the male connector part is received by (e.g. in) the female connector part to form a sealed joint in which at least part of the first flange deforms against the sealing surface, the one or more radially inwardly projecting members are disposed in the annular recess between the first flange and the second flange, and an outer surface of the second flange is radially outward of at least part of the one or more radially inwardly projecting members; and

wherein in the sealing configuration fluid may flow through the bore from a first side of the sealed joint to a second side of the sealed joint.

In the sealing configuration, the at least part of the first flange may form a sealed interference fit with the sealing surface.

In certain embodiments, the one or more radially inwardly projecting members comprise a plurality of radially flexible fingers.

In certain embodiments, the outer surface of the second flange may narrow relative to a longitudinal axis of the sealable joint.

In certain embodiments, the second flange may comprise an abutment surface that faces the one or more radially inwardly projecting members in the sealing configuration, wherein abutment between the abutment surface and the one or more radially inwardly projecting members may limit axial movement of the second flange relative to the third component.

In certain embodiments, the male connector part may comprise a third flange, wherein the third flange is on a side of the first flange that is opposite the second flange, and wherein abutment between the third flange and the female connector part may limit axial movement of the first component relative to the third component.

In accordance with another aspect of the present invention, there is provided an autoinjector sub-assembly comprising a sealable joint as described above.

The autoinjector sub-assembly may comprise a propellant source, wherein the sealable joint may seal the propellant source to another component of the auto-injector sub-assembly. The propellant source may comprise a propellant housing defining a reservoir for containing a propellant, and the male connector part may be moveable relative to the propellant housing so as to selectively put the bore in fluid communication with the reservoir.

The female connector part may form at least part of the other component of the auto-injector sub-assembly.

In accordance with another aspect of the present invention, there is provided a propellant source comprising a propellant housing defining a reservoir for containing a propellant, a male connector part comprising a stem having a bore therethrough, and comprising a first flange and a second flange that define an annular recess therebetween, and wherein the male connector part is moveable relative to the propellant housing so as to selectively put the bore in fluid communication with the reservoir.

In certain embodiments, the outer surface of the second flange may narrow relative to a longitudinal axis of the propellant source.

In certain embodiments, the male connector part may comprise a third flange, wherein the third flange is on a side of the first flange that is opposite the second flange.

In accordance with an aspect of the present invention, there is provided a propellant source for containing and dispensing a propellant, comprising:

a housing defining a reservoir for containing the propellant; and

a stem extending through an opening in the housing and having a bore that extends through the stem and has an outlet, and one or more radial channels extending from the bore through an external surface of the stem;

wherein the stem is axially moveable relative to the housing between a first axial position in which the one or more radial channels are not in fluid communication with the reservoir, and a second axial position in which the one or more radial channels are in fluid communication with the reservoir such that propellant may flow from the reservoir, through the one or more radial channels, through the bore and out of the outlet; and

wherein the stem comprises a first stem part and a second stem part connected to the first part, the second stem part being disposed entirely within the housing and having a width that is greater than a diameter of the opening so as to retain part of the stem in the housing.

In certain embodiments, a rear end of the first stem part extends through a bore of the second stem part and comprises a radially extending flange portion that has a width greater than a diameter of the bore of the second stem part and prevents axially downward movement of the first stem part relative to the second stem part. In certain embodiments, the second stem part comprises one or more axial holes therethrough. The one or more axial holes may reduce the low pressure effect created between the second stem part and a seal of the propellant source.

In accordance with an aspect of the present invention, there is provided a propellant source for containing and dispensing a propellant, comprising:

a housing defining a reservoir for containing the propellant; and

a stem extending through an opening in the housing and having a bore that extends through the stem and has an outlet, and one or more radial channels extending from the bore through an external surface of the stem;

wherein the stem is axially moveable relative to the housing between a first axial position in which the one or more radial channels are not in fluid communication with the reservoir, and a second axial position in which the one or more radial channels are in fluid communication with the reservoir such that propellant may flow from the reservoir, through the one or more radial channels, through the bore and out of the outlet;

wherein a part of the stem that is disposed entirely within the housing has a width that is greater than a diameter of the opening so as to retain part of the stem in the housing, and wherein said part of the stem comprises one or more axial holes therethrough. The one or more axial holes may reduce the low pressure effect created between the second stem part and a seal of the propellant source.

In accordance with an aspect of the present invention, there is provided a method of manufacturing the propellant source described above, wherein the method comprises assembling the first stem part and second stem part together and forming the radially extending flange portion of the first stem part by mechanically deforming the first stem part against the second stem part. In certain embodiments, the step of mechanically deforming the first stem part may comprise heat staking (e.g. the rear end of) the first stem part.

### BRIEF DESCRIPTION OF THE DRAWINGS

Embodiments of the invention are further described hereinafter with reference to the accompanying drawings, in which:

FIG. 1 is a cross-sectional view of a sealable joint according to an embodiment of the present invention;

FIG. 2 is a further cross-sectional view of the sealable joint of FIG. 1 additionally showing a flexed position of the one or more radially inwardly projecting members;

FIG. 3 is a further cross-sectional view of the sealable joint of FIG. 1 additionally showing potential dimension variations within tolerance limits;

FIG. 4 shows a part of an autoinjector sub-assembly that includes a sealable joint in accordance with an embodiment of the present invention;

FIG. 5 schematically shows an autoinjector device that includes the autoinjector sub-assembly of FIG. 4;

FIG. 6A shows two parts of a stem of a propellant source in accordance with an embodiment of the present invention, prior to completion of manufacturing;

FIG. 6B shows the two parts of the stem of FIG. 6B after completion of manufacturing; and

FIG. 7 is a cross-sectional view of a sealable joint according to an alternative embodiment of the present invention.

### DETAILED DESCRIPTION

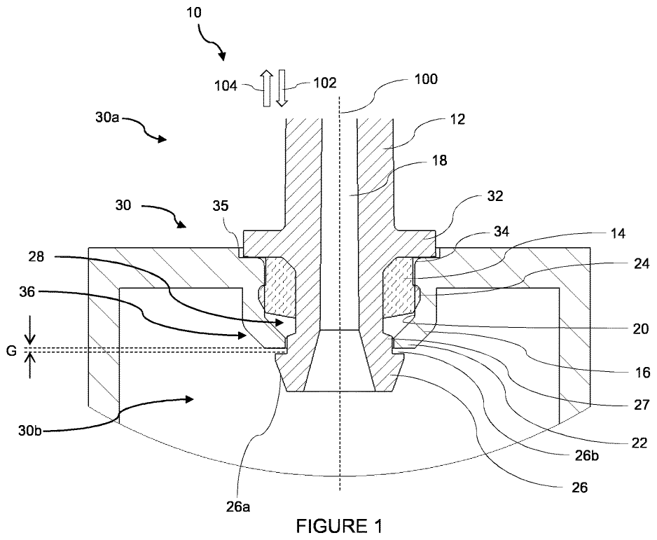

A sealable joint 10 in accordance with an embodiment of the present invention is shown in FIG. 1 in cross-section. In certain embodiments, the sealable joint 10 seeks to create an externally fluid-tight seal that is also mechanically robust, and that permits passage of a fluid through a bore from one side of the fluid-tight seal to another side of the fluid-tight seal. The sealable joint 10 includes a first component 12 that comprises a stem. The first component 12 has a bore 18 therethrough that permits the passage of a fluid through the first component 12. The sealable joint 10 extends along a longitudinal axis 100 along which the bore 18 is centred and runs parallel to.

Throughout the present specification, all directions that are referred to as axial or similar, are intended to mean directions that are along or parallel to the longitudinal axis 100. All directions that are referred to as circumferential or similar, are intended to mean directions along the arc of a notional circle that is centred on the longitudinal axis 100 and whose plane is perpendicular to the longitudinal axis 100. All directions that are referred to as radial or similar, are intended to mean directions that extend away from the longitudinal axis 100 and are perpendicular to the longitudinal axis 100. A point that is radially outward relative to another point is further from the longitudinal axis 100 than that other point.

In the embodiments shown in the Figures, a second component 14 is disposed on the first component 12. In certain embodiments, the second component 14 may be affixed to the first component 12, e.g. by an adhesive or by frictional engagement. In certain embodiments, the second component may be moulded onto or over the first component 12. In certain embodiments, the second component 14 may comprise a more resilient material relative to the material of the first component 12. n some alternative embodiments, the second component 14 may be made of the same material (or at least a material having the same resilience) as the first component 12. In this sense, the first component 12 and the second component 14 may form a single unitary part, albeit with distinct identifiable regions and/or with distinct functional characteristics. This single unitary part may be formed as a single part, or it may be formed as multiple parts that are subsequently fused together.

In the non-limiting embodiment shown in FIG. 1, the second component 14 comprises a first flange 24, and the first component 12 comprises a second flange 26. The first flange 24 and second flange 26 are each radially extending parts of the second component 14 and first component 12, respectively. In certain embodiments, the first flange 24 is circumferentially continuous whilst the second flange 26 may be either circumferentially continuous or discontinuous. In certain embodiments, the first flange 24 may be an o-ring seal or a lip seal. The first flange 24 and the second flange 26 are axially spaced from one another such that, together, they define an annular recess 28 therebetween. In alternative embodiments, the first flange 24 and second flange 26 of the sealable joint 10 may be formed by other components. In particular, one of the first component 12 and the second component 14 may comprise the first flange 24 and the second flange 26, or a combination of the first component 12 and the second component 14 may comprise the first flange 24 and the second flange 26.

A third component 16 is provided and comprises a sealing surface 20 and one or more radially inwardly projecting members 22. In certain embodiments, the one or more radially inwardly projecting members 22 may extend radially inwards towards the longitudinal axis 100 at an angle that is preferably not 90°. In embodiments in which the one or more radially inwardly projecting members 22 extend radially inwards towards the longitudinal axis 100 at an angle that is not 90°, the one or more radially inwardly projecting members 22 may additionally extend in a direction that is parallel to direction 102. In such embodiments, the one or more radially inwardly projecting members 22 may be more resilient to upward axial loading in contrast to alternative arrangements. In embodiments in which the third component 16 comprises a single radially inwardly projecting member 22 (as shown in FIG. 1), the radially inwardly projecting member 22 may be a circumferentially continuous annulus. In other embodiments, more than one radially inwardly projecting member 22 may be provided, e.g. in the form of a plurality of radially inwardly extending fingers. In either case, the one or more radially inwardly projecting members 22 extend radially inwards only insofar as an aperture remains therebetween, wherein the aperture may permit the passage of the second flange 26 therethrough. In this sense, the first component 12 and second component 14 may be considered to collectively form a male connector part (whether or not they form a single unitary component), whilst the third component 16 may be considered to form a female connector part that is capable of receiving (part of) the male connector part.

FIG. 1 shows the first component 12, the second component 14 and the third component 16 in a sealing configuration. To establish the sealing configuration, the first component 12 (with the second component 14 disposed on the first component 12) is moved axially downwards relative to the third component 16. The axially downward direction is indicated by arrow 102 in FIG. 1. In doing so, the second flange 26 contacts the one or more radially inwardly projecting members 22 and causes them to flex or otherwise deform radially outwardly to permit the second flange 26 to pass. To facilitate passage of the second flange 26, the second flange is provided with a narrowing outer surface 26a that may act as a cam against the one or more radially inwardly projecting members 22 and cause radial flexing or deformation thereof. Once the second flange 26 has axially passed the one or more radially inwardly projecting members 22, the one or more radially inwardly projecting members 22 can flex or deform radially inwardly back towards their original (or normal) radial positions (i.e. their positions prior to being radially flexed or otherwise deformed). In certain embodiments, the one or more radially inwardly projecting members 22 may not return completely to their original radial positions due to interference with the first component 12 or the second component 14 (and also because the material might be permanently deformed by the insertion).

FIG. 1 shows the sealable joint 10 in the sealing configuration in which the second flange 26 has axially passed the one or more radially inwardly members 22, and the one or more radially inwardly members 22 have returned to their original radial positions. A widened portion 27 of the first component 12 is provided axially above the second flange 26 and has a radius that is similar to the radius defined by an innermost edge of the one or more radially inwardly projecting members 22. In the configuration shown in FIG. 1, the one or more radially inwardly projecting members 22 are adjacent to the widened portion 27, and due to their relative radii, no significant gap exists between the widened portion 27 and the one or more radially inwardly projecting members 22. This close fitting limits relative transverse movement between the first component 12 and the third component 16. In the sealing configuration, the one or more radially inwardly projecting members 22 are disposed in the annual recess 28 between the first flange 24 and the second flange 26, and the outer surface 26a of the second flange 26 is radially outward of at least part of the one or more radially inwardly projecting members 22. As a result, upward axial movement (indicated by arrow 104 in FIG. 1) of the first component 12 relative to the third component 16 is limited (such that further axially upward movement is prohibited) by abutment of the second flange 26 and the one or more radially inwardly members 22. In the particular embodiments shown in FIG. 1, a rearward (i.e. upwardly facing in the Figures) axial surface 26b (or abutment surface) of the second flange 26 has a profile that reduces the risk of the rearward axial surface 26b acting as a cam against the one or more radially inwardly projecting members 22 in the event that the first component 12 was urged in the upward axial direction 104 relative to the third component 16.

A third flange 32 is formed on the first component 12 and is provided axially upwards of the first flange 24 (i.e. on the opposite side of the first flange 24 than the second flange 26). The third flange 32 extends radially outwardly to a radius that is greater than the radius of an aperture defined in the third component 16 by a top shoulder 34 that is axially upwards of the sealing surface 20 and the one or more radially inwardly projecting members 22. The relative profiles of the third flange 32 and top shoulder 34 are such that when they engage one another, camming effects are minimized. Rather, engagement of the third flange 32 with the top shoulder 34 prevents further downward axial movement (i.e. along direction 102) of the first component 12 relative to the third component 16. Therefore, once in the sealing configuration, the male connector part is engaged with the female connector part and disengagement of the male connector part from the female connector part is prevented by engagement of the second flange 26 with the one or more radially inwardly projecting members 22 along direction 104 and by engagement of the third flange 32 with the top shoulder 34 along direction 102. Thus, once in the sealing configuration, the male connector part is mechanically retained by the female connector part.

In the sealing configuration, the second component 14 is disposed between the first component 12 and the third component 16 such that the first flange 24 is caused to deform against the sealing surface 20 of the third component 22. This occurs because the first flange 24 nominally extends radially outwards of the sealing surface 20. The deformed first flange 24 forms a fluid-tight seal with the sealing surface 20. Thus, in addition to the mechanical retention of the male connector part relative to the female connector part, a sealed joint 30 is provided that, with the exception of fluid through the bore 18, substantially prevents the flow of fluid across the sealed joint 30 between (in both directions) a first side 30a of the sealed joint 30 and a second side 30b of the sealed joint 30. In order for the first flange 24 to deform and provide a seal against the sealing surface 20, sufficient surrounding space must be provided to enable the deformation. In the embodiment shown in FIG. 1, the annular recess 28 provides sufficient space for the second component 14 to deform into so that the first flange 24 can deform against the sealing surface 20 and provide a fluid-tight seal.

A sealed joint 10 in accordance with embodiments of the present invention may provide a robust mechanical connection and fluidic seal. In certain embodiments, the sealed joint 10 may be formed by a simple snap fit connection. By virtue of the features described above, the sealed joint 10 may maintain its mechanical and sealing integrity under conditions such as vibration (sinusoidal and/or random), pressure loading, axial loading, transverse loading, and/or shock loading. Furthermore, given that the assembled sealed joint 10 includes interfaces that abut or interfere with one another so as to prevent or limit relative movement in each of the up, down and radial directions, the sealed joint 10 prevents (or at least significantly reduces the risk of) incorrect assembly. Certain embodiments may provide an “anti-tamper” joint that may require specialist tools and/or a specialist method to dismantle.

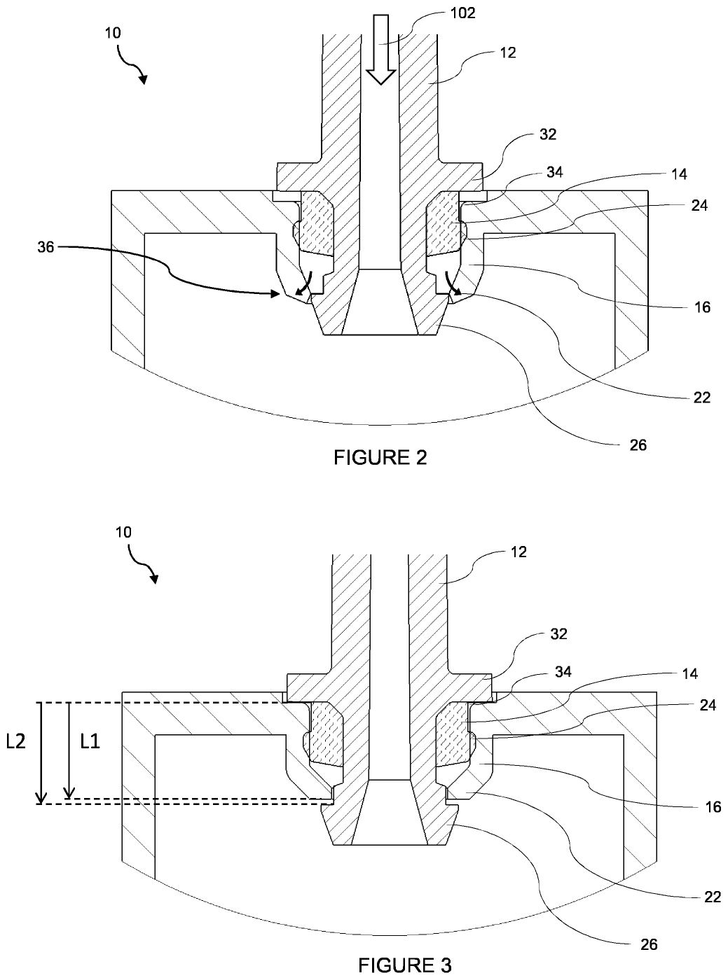

The sealed joint 10 is shown again in FIG. 2, where the one or more radially inwardly projecting members 22 are shown in a deflected position. The one or more radially inwardly projecting members 22 may deflect radially to (or through) the position shown in FIG. 2 during assembly as the second flange 26 urges the one or more radially inwardly projecting members 22 radially outwardly in order to pass. As seen from FIG. 2, an end(s) of the one or more radially inwardly projecting members 22 moves along an arc when deflecting radially outwardly. As such, the end(s) of the one or more radially inwardly projecting members 22 additionally moves axially downwardly as it moves radially outwardly. Therefore, in order to relax back to or towards its original radial position, it must move axially upwardly in addition to radially inwardly. To permit this travel, the male connector part (the first component 12 in the specific embodiment shown in FIGS. 1 to 3) must have a form and position that provides the necessary space for the required movement of the one or more radially inwardly projecting members 22. Returning to FIG. 1, it can be seen that when the first component 12 is in its maximum downward axial position relative to the third component 16 (as determined by abutment between the third flange 32 and top shoulder 34), a gap G exists between the one or more radially inwardly projecting members 22 and second flange 26. It is this gap G that is required to be sufficient to permit the swinging (i.e. axial movement in addition to radial movement) of the one or more radially inwardly projecting members 22 in order for the one or more radially inwardly projecting members 22 to relax back to or towards their original radial positions once the second flange 26 has passed therethrough.

Referring to FIGS. 1 and 3, the gap G is determined by the relative dimensions L1 and L2, where L1 is the axial distance between the top shoulder 34 and the bottom of the one or more radially inwardly projecting members 22, and L2 is the axial distance between the bottom of the third flange 32 and the top of the second flange 26, and G=L2−L1. Given that manufacturing tolerances mean that the actual lengths L1 and L2 may vary in a manufactured sealed joint 10 relative to the nominal lengths intended, the gap G will also vary across a batch of manufactured sealed joint 10 products. For example, L1 may be longer than the nominal length and L2 may be shorter than the nominal length. Such variation can be expected within manufacturing tolerances. The magnitude of the nominal gap G should therefore be chosen such that, even if L1 and L2 are at their extreme magnitudes due to tolerance variations, the one or more radially inwardly projecting members 22 are able to flex radially outwardly and axially downwardly in order to permit the second flange 26 to pass during assembly, and flex radially inwardly and axially upwardly thereafter so that at least part of the one or more radially inwardly projecting members 22 is radially inwards of the outer surface 26a of the second flange 26.

Additionally, the components surrounding the one or more radially inwardly projecting members 22 provide an outer annular space 36 that provides the necessary space for the one or more radially inwardly projecting members 22 to radially flex or deform into to permit assembly of the sealed joint 10.

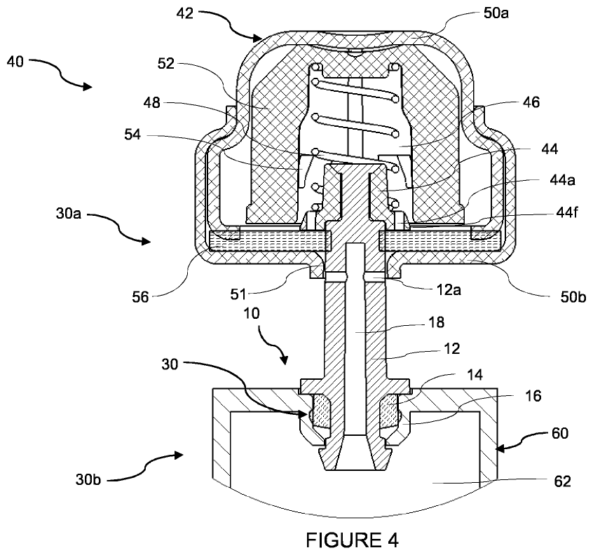

FIG. 4 shows a part of an autoinjector sub-assembly 40 comprising a propellant source 42 and a cylindrical housing 60. The propellant source 42 is connected to the cylindrical housing 60 by the sealable joint 10. In particular, the first component 12 forms a first stem part of the propellant source 42, while the third component 16 forms a part of the cylindrical housing 60.

The propellant source 42 may comprise or share features with the valved dispenser described in WO2013182856 (Consort Medical Plc), but incorporate the features described above in relation to the male connector part of the sealable joint 10.

The propellant source 42 of FIG. 4 comprises a propellant housing 50 that is formed from a first propellant housing part 50a and a second propellant housing part 50b. The propellant housing 50 surrounds and defines an internal reservoir 46 that is capable of containing a propellant, such as a liquefied gas. The first component 12 (first stem part) extends into the reservoir 46 through an opening 51 of the propellant housing 50 and may sealingly slide relative thereto. A seal 56 is provided to seal against the first component 12 yet permit it to slide into and out of the reservoir 46. A second stem part 44 is connected to the first component (first stem part) in the region that is contained within the propellant housing 50 (as described in more detail below). As such, the first component 12 and the second stem part 44 collectively form a stem. The second stem part 44 is contained entirely within the propellant housing 50. The second stem part 44 flares radially outwardly at a flange portion 44f so as to prevent the first component 12 (first stem part) from exiting the propellant housing 50 completely. In particular, the flange portion 44f of the second stem part 44 is dimensioned so as to have a width that is greater than the diameter of the opening 51 of the propellant housing 50 through which the first component 12 extends. Given that the first component 12 and the second stem part 44 are connected to one another, the flange portion 44f limits downward movement of the second stem part 44 (and first component 12) relative to the propellant housing 50, thus retaining the second stem part 44 (and first component 12) in the propellant housing 50.

The first component 12 includes radial channels 12a that extend radially from the bore 18 through the external surface of the first component 12. In a first axial position of the first component 12 relative to the propellant housing 50, the radial channels 12a are disposed below the seal 56 so that the bore 18 is not in fluid communication with the reservoir 46. The first component 12 may move axially upwardly relative to the propellant housing 50 to a second axial position in which the radial channels 12a are disposed above the seal 56 and are in fluid communication with the reservoir 46. Therefore, in the second axial position, the bore 18 is in fluid communication with the reservoir 46 and propellant from the reservoir 46 may pass into and through the bore 18 (via the radial channels 12a) and out of an outlet of the bore 18 from the first side 30a of the sealed joint 30 to the second side 30b of the sealed joint 30.

In the non-limiting embodiment shown in FIG. 4, ribs 52 are provided within the propellant housing 50 and act as a reaction surface and retainer for a spring 48. In the non-limiting embodiment of FIG. 4, the ribs 52 are not integral with the propellant housing 50 (in contrast to certain prior art arrangements such as WO2013182856A1). In certain embodiments, the ribs 52 may not be present at all. The spring 48 acts on the second stem part 44 to bias the first component 12 (first stem part) towards the first axial position. The spring force of the spring 48 must be acted against and overcome for the first component 12 to move to the second axial position. Latches 54 extend from the boss 52 axially upwardly and radially inwardly. The latches 54 are configured such that they may deflect radially outwardly to permit the second stem part 44 to pass as the first component 12 is moved axially upwardly relative to the propellant housing 50. When the second stem part 44 is axially above the latches 54, the latches 54 may relax back to or towards their radially inward positions (that are radially inwards of at least part of the second stem part 44), thus preventing subsequent downward axial movement of the second stem part 44 (and first component 12) relative to the propellant housing 50. Thus, once the second stem part 44 has been moved into the propellant housing 50 by a sufficient amount, the latches 54 latch the second stem part 44 and retain the first component 12 in the second axial position. The second stem part 44 includes one or more axial channels 44a that permit the passage of fluid across the second stem part 44 so as to reduce any low pressure effect between the second stem part 44 and the seal 56 (which would increase the activation force) when the first component 12 moves from the first axial position to the second axial position. In alternative embodiments, the propellant source 42 may not comprise any latching means for retaining the first component 12 in the second axial position.

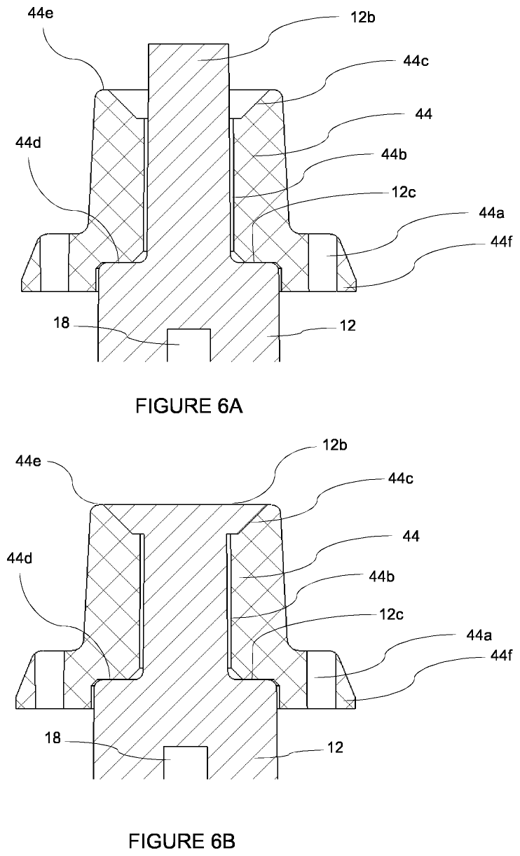

FIGS. 6A and 6B illustrate a method of forming the connection between the first stem part 12 and the second stem part 44. FIG. 6A shows the first stem part 12 and the second stem part 44 during (and before completion of) manufacture, whilst FIG. 6B shows the first stem part 12 and the second stem part 44 after manufacture is complete. A rear end 12b of the first stem part 12 extends through a bore 44b of the second stem part 44 and further extends through a first recess 44c of the second stem part 44. In order to fixedly connect the first stem part 12 to the second stem part 44, the rear end 12b is mechanically deformed against the second stem part 44 so as to create a radially extending flange portion that has a width greater than a diameter of the bore 44b of the second stem part 44 and prevents axially downward movement of the first stem part 12 relative to the second stem part 44. In the embodiment shown in FIGS. 6A and 6b, the rear end 12b is mechanically deformed into the recess 44c of second stem part 44 so that, when deformed, the rear end 12b of the first stem part 12 is substantially flush with a rear end 44e of the second stem part 44. Suitable methods of mechanical deformation include, but are not limited to, heat staking and ultrasonic staking. In alternative embodiments, the first stem part 12 and second stem part 44 may be connected to one another without permanent deformation of one of the parts. For example, a snap fit connection may connect the first stem part 12 and second stem part 44 together.

Additionally, the first stem part 12 has a shoulder portion 12c that extends radially outwardly relative to the portion of the first stem part 12 that extends through the bore 44b. Abutment between the shoulder portion 12c and the second stem part 44 limits upward movement of the first stem part 12 relative to the second stem part 44. In the non-limiting embodiment of FIGS. 6A and 6B, the shoulder portion 12c resides in a second recess 44d of the second stem part 44. As such, lateral movement of the first stem part 12 relative to the second stem part 44 is also limited by abutment therebetween. The connection formed between the first stem part 12 and second stem part 44 mechanically fixes the two parts to one another. The connection may not necessarily (and is not required to) form a hermetic seal between the two parts. As such, in use, propellant may flow along the interface between the first stem part 12 and the second stem part 44.

The first stem part 12 and/or the second stem part 44 are preferably made from a strong material that is substantially impermeable to liquefied propellant (such as HFA). In certain embodiments, either or both of the first stem part 12 and the second stem part 44 are made of glass filled polybutylene terephthalate (PBT). Other suitable materials include materials that are substantially impermeable to propellants such as HFA.

The cylindrical housing 60 may receive propellant passing through the bore 18 from the propellant source 42. In the embodiment shown in FIG. 4, the propellant exits the bore 18 and enters a receiving chamber 62. The propellant may exit the propellant source in a liquid phase and boil outside of the propellant source to produce a vapour pressure. In alternative embodiments, the propellant may exit the propellant source as a gas. In any embodiment, propellant received in the receiving chamber 62 causes the vapour pressure in the receiving chamber 62 to rise. The vapour pressure will naturally act on the sealable joint 10. The sealable joint 10 of the present invention may resist such rising pressures such that its integrity is not compromised as a result.

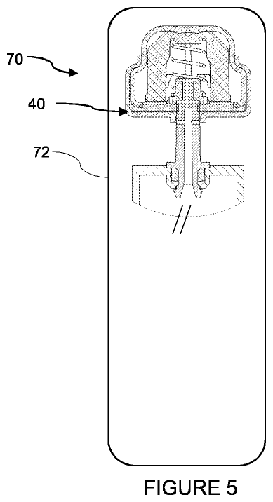

FIG. 5 schematically shows an autoinjector device 70 that comprises an outer housing 72 that contains the autoinjector sub-assembly 40 described above.

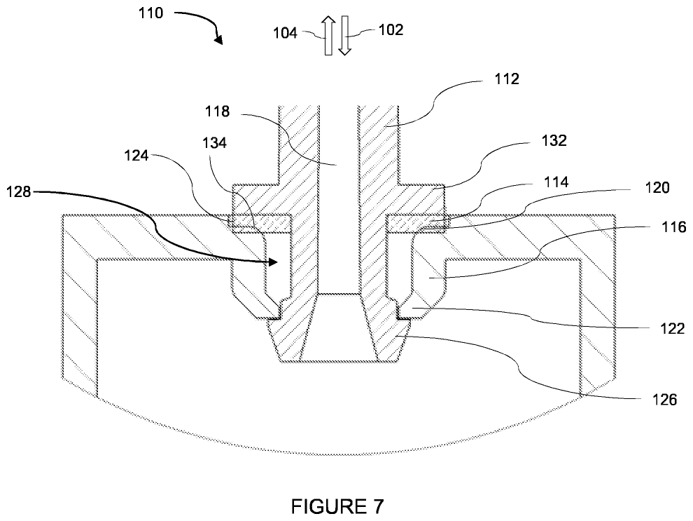

A sealable joint 110 in accordance with an alternative embodiment of the present invention is shown in FIG. 7 in cross-section. The sealable joint 110 shares many features with the embodiments described above and corresponding, alike or otherwise similarly functioning components are indicated using the same reference numerals, but transposed by 100. Characteristics described above in respect of certain features may apply to the related (i.e. as indicated by a transposed reference numeral) features of the embodiment of FIG. 7.

The sealable joint 110 includes a first component 112 that comprises a stem. The first component 112 has a bore 118 therethrough that permits the passage of a fluid through the first component 112.

A second component 114 is disposed on the first component 112. The second component 114 comprises a first flange 124, and the first component 112 comprises a second flange 126. The first flange 124 and second flange 126 are each radially extending parts of the second component 114 and first component 112, respectively. In certain embodiments, the first flange 124 is circumferentially continuous whilst the second flange 126 may be either circumferentially continuous or discontinuous. In certain embodiments, the first flange 124 may be an o-ring or gasket seal . The first flange 124 and the second flange 126 are axially spaced from one another such that, together, they define an annular recess 128 therebetween.

A third component 116 is provided and comprises a sealing surface 120 and one or more radially inwardly projecting members 122. The one or more radially inwardly projecting members 122 extend radially inwards only insofar as an aperture remains therebetween, wherein the aperture may permit the passage of the second flange 126 therethrough. In this sense, the first component 112 and second component 114 may be considered to collectively form a male connector part (whether or not they form a single unitary component), whilst the third component 116 may be considered to form a female connector part that is capable of receiving (part of) the male connector part.

A third flange 132 is formed on the first component 112 and is provided axially upwards of the first flange 124 (i.e. on the opposite side of the first flange 124 than the second flange 126). The third flange 132 extends radially outwardly to a radius that is greater than the radius of an aperture defined in the third component 116 by a top shoulder 134 that is axially upwards of the sealing surface 120 and the one or more radially inwardly projecting members 122. The relative profiles of the third flange 132 and top shoulder 134 are such that when they engage one another, camming effects are minimized. Rather, engagement of the third flange 132 with the top shoulder 134 prevents further downward axial movement (i.e. along direction 102) of the first component 112 relative to the third component 116. Therefore, once in the sealing configuration, the male connector part is engaged with the female connector part and disengagement of the male connector part from the female connector part is prevented by engagement of the second flange 126 with the one or more radially inwardly projecting members 122 along direction 104 and by engagement of the third flange 32 with the top shoulder 34 along direction 102. Thus, once in the sealing configuration, the male connector part is mechanically retained by the female connector part.

In the sealing configuration, the second component 114 is disposed between the third flange 132 and the third component 116 such that the first flange 124 is caused to deform against the sealing surface 120 of the third component 122. The deformed first flange 124 forms a fluid-tight seal with the sealing surface 120. Thus, a sealed joint is provided in a similar manner to the sealed joint 30 described above.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The readers attention is directed to all papers and documents which are filed concurrently with or previous to this specification in connection with this application and which are open to public inspection with this specification, and the contents of all such papers and documents are incorporated herein by reference.

1. A sealable joint comprising:
a first component, a second component, and a third component;
the first component comprising a stem having a bore therethrough for the passage of a fluid;
the second component being disposed on the first component; and
the third component comprising a sealing surface and one or more radially inwardly projecting members; and
wherein one of the second component and the first component, or a combination of the second component and the first component comprise a first flange and a second flange that define an annular recess therebetween;
wherein in a sealing configuration, the second component is received by the third component to form a sealed joint in which at least part of the first flange deforms against the sealing surface, the one or more radially inwardly projecting members are disposed in the annular recess between the first flange and the second flange, and an outer surface of the second flange is radially outward of at least part of the one or more radially inwardly projecting members; and
wherein in the sealing configuration fluid may flow through the bore from a first side of the sealed joint to a second side of the sealed joint.

2. The sealable joint according to claim 1, wherein the second component is affixed on an external surface of the first component.

3. The sealable joint according to claim 1, wherein the second component comprises a more resilient material relative to the first component.

4. The sealable joint according to claim 1, wherein in the sealing configuration, the at least part of the first flange forms a sealed interference fit with the sealing surface.

5. The sealable joint according to claim 1, wherein the one or more radially inwardly projecting members comprise a plurality of radially flexible fingers.

6. The sealable joint according to claim 1, wherein the outer surface of the second flange narrows relative to a longitudinal axis of the sealable joint.

7. The sealable joint according to any preceding claim 1, wherein the second flange comprises an abutment surface that faces the one or more radially inwardly projecting members in the sealing configuration, wherein abutment between the abutment surface and the one or more radially inwardly projecting members limits axial movement of the second flange relative to the third component.

8. The sealable joint according to claim 1, wherein the first component comprises a third flange, wherein the third flange is on a side of the first flange that is opposite to the second flange such that the first flange is between the second flange and the third flange, and wherein abutment between the third flange and the third component limits axial movement of the first component relative to the third component.

9. The sealable joint according to claim 1, wherein the first component and the second component form a single unitary part.

10. An autoinjector sub-assembly comprising:
a sealable joint comprising a first component, a second component, and a third component
the first component comprising a stem having a bore therethrough for the passage of a fluid:
the second component being disposed on the first component; and
the third component comprising a sealing surface and one or more radially inwardly projecting members; and
wherein one of the second component and the first component, or a combination of the second component and the first component comprise a first flange and a second flange that define an annular recess therebetween;
wherein in a sealing configuration, the second component is received by the third component to form a sealed joint in which at least part of the first flange deforms against the sealing surface, the one or more radially inwardly projecting members are disposed in the annular recess between the first flange and the second flange, and an outer surface of the second flange is radially outward of at least part of the one or more radially inwardly projecting members; and
wherein in the sealing configuration fluid may flow through the bore from a first side of the sealed joint to a second side of the sealed joint.

11. The autoinjector sub-assembly according to claim 10, comprising a propellant source, wherein the sealable joint seals the propellant source to another component of the auto-injector sub-assembly.

12. The autoinjector sub-assembly according to claim 11, wherein the propellant source comprises a propellant housing defining a reservoir for containing a propellant, and the first component is moveable relative to the propellant housing so as to selectively put the bore in fluid communication with the reservoir.

13. The auto-injector sub-assembly according to claim 11, wherein the third component forms at least part of the other component of the auto-injector sub-assembly.

14. A propellant source comprising a propellant housing defining a reservoir for containing a propellant, a first component comprising a stem having a bore therethrough, and a second component being disposed on the first component, wherein one of the second component and the first component, or a combination of the second component and the first component comprise a first flange and a second flange that define an annular recess therebetween, and wherein the first component is moveable relative to the propellant housing so as to selectively put the bore in fluid communication with the reservoir.
